(19) United States
(12) Patent Application Publication
(10) Pub. No.: US 2021/0052764 A1
(43) Pub. Date: Feb. 25, 2021

(54) AIR STERILIZER UNIT

(71) Applicant: DOLPHIN CARE APS, Hørsholm (DK)

(72) Inventor: Jørn TERKELSEN, Hørsholm (DK)

(73) Assignee: DOLPHIN CARE APS, Hørsholm (DK)

(21) Appl. No.: 16/968,971

(22) PCT Filed: Feb. 13, 2019
(86) PCT No.: PCT/EP2019/053479

§ 371 Date: Aug. 11, 2020

(30) Foreign Application Priority Data
Feb. 14, 2018 (EP) 18156783.5

(51) Int. Cl. A61L 9/20

(52) U.S. Cl. CPC A61L 9/205; A61L 2209/12; A61L 2209/14

(57) ABSTRACT

The present invention relates to air sterilizer unit having at least one inlet and at least one outlet comprising (a) a filter housing having an interior space wherein at least one UCV light source adapted for radiating microorganisms, is located, and at least one filter with a capacity to reduce or remove microorganisms from ambient air, wherein the filter is UVC resistant and is located between the UVC light source and the at least one outlet, (b) an air transportation unit, wherein the air transportation unit is adapted to move ambient air from the at least one inlet into the filter housing, and being exposed to the UVC light and through the at least one filter, and through the at least one outlet.

### FIELD OF THE INVENTION

The present invention relates to an air sterilizer unit for sterilizing ambient air is for instance a hospital facility. Furthermore, the present invention is concerned with a method for removing microorganisms and other polluting particles from ambient air, such as air in a hospital facility. The present invention also relates to use of such air sterilizer unit for cleaning ambient air.

### BACKGROUND OF THE INVENTION

The air in buildings and in particular rooms in buildings, such as rooms in a hospital is strongly influenced by the surroundings and the environment where polluted air is all over such buildings and in the rooms. With today's air conditioners and other air cleaning devices used indoor, the efficiency in reducing odors, and other polluting objects, including microorganisms, is poor and removing such objects and microorganisms is not possible.

### SUMMARY OF THE INVENTION

The present inventors have realized that there is a need for improving air cleaning at hospital facilities, and in particular, hospital rooms for housing sick people, and rooms for surgery. Similar needs, without limitation, are present when it comes to nurseries, kinder gardens, homes for old people, educational facilities, offices, production facilities. The ambient air at such places is polluted with microorganisms, such as bacteria, virus, fungus, and also with other particles that smells and create a poor indoor environment. The present invention is capable of reducing such odors and at the same time reduce microorganisms, and in preferred embodiments is capable of removing both orders and microorganisms from the ambient air indoor.

The present invention concerns an air sterilizer unit having at least one inlet and at least one outlet comprising
(a) a filter housing having an interior space wherein at least one UVC light source adapted for radiating microorganisms, is located, and at least one filter with a capacity to reduce or remove air borne particles and microorganisms from ambient air, wherein the filter is UVC resistant and is located between the UVC light source and the at least one outlet,
(b) an air transportation unit, wherein the air transportation unit is adapted to move ambient air from the at least one inlet into the filter housing and being exposed to the UVC light and through the at least one filter, and through the at least one outlet.

In one embodiment of the present invention the air sterilizer unit wherein a pre-filter capable of removing air borne particles having a diameter less than 200 μm is located at the inlet. Preferably, the pre-filter is capable of removing air borne particles having a diameter less than 100 μm.

In a further embodiment of the present invention the air transportation unit comprises a fan having sufficient capacity to move ambient air into the filter housing and through the at least one filter and the at least one outlet.

In a further embodiment of the present invention the UVC light source is adapted to provide UVC light at 250-260 nm, such as 254 nm.

In a further embodiment of the present invention the at least one filter is a HEPA filter capable of removing air borne particles having a diameter less than 0.5 μm. Preferably, the HEPA filter is a HEPA14 filter capable of removing at least 99,995% of air borne particles 0.3 micrometers (μm) in diameter.

In a further embodiment of the present invention a second filter which is UVC resistant is located between the UVC light source and the at least one filter and comprises an active carbon filter capable of removing air borne particles having a diameter less than 100 μm.

In a further embodiment of the present invention a third filter which is UVC resistant is located between the UVC light source and the second filter and capable of removing air borne particles having a diameter less than 100 μm, wherein the filter is coated with TiO2.

In a further embodiment of the present invention the filter housing is a cylinder shaped housing. Typically, the air sterilizer unit is adapted to draw ambient air into the air transportation unit and into the filter housing to receive radiation by the UVC light and out through the at least one outlet located at the side of the cylinder housing.

In a further embodiment of the present invention the UVC light source comprises between 1 and 10 UVC lamps, such as 3 or 4 UVC lamps.

In a further embodiment of the present invention the HEPA filter is folded, such as pleated, to create a large surface area.

In a further embodiment of the present invention the air sterilizer unit is a stand-alone device.

In a further embodiment of the present invention the air sterilizer unit as a stand-alone device is suitable for functioning inside a closed room.

In another aspect the present invention relates to a method of removing microorganisms from ambient air comprising supplying current to the air sterilizer unit of the present invention and moving ambient air from the at least one inlet into the filter housing, wherein the air is being exposed to the UVC light and further moving the air exposed to UVC light through the at least one filter, and through the at least one outlet, wherein microorganisms have been removed from the air leaving the at least one outlet.

In a further aspect the present invention relates to use of an air sterilizer unit of the present invention in a room, for removing microorganisms from the room.

The present invention provides these advantages with the described solution.

Further objects and advantages of the present invention will appear from the following description, and claims.

### DESCRIPTION OF THE INVENTION

The present invention concerns an air sterilizer unit having at least one inlet and at least one outlet comprising

(a) a filter housing having an interior space wherein at least one UVC light source adapted for radiating microorganisms, is located, and at least one filter with a capacity to reduce or remove air borne particles and microorganisms from ambient air, wherein the filter is UVC resistant and is located between the UVC light source and the at least one outlet,

(b) an air transportation unit, wherein the air transportation unit is adapted to move ambient air from the at least one inlet into the filter housing and being exposed to the UVC light and through the at least one filter, and through the at least one outlet.

The air sterilizer unit of the present invention is typically located inside a metal mesh to protect the device form injuries such as people by accident pushing or hitting the device. Also, the device is adapted to be connected to current in order to function in a house, such as a hospital, in particular a hospital room. Although the air sterilizer unit is described as two parts (a) and (b) it can come as separate parts which may then be connected at the relevant facility, or the air transportation unit may be housed at the relevant facility and the filter housing may be exchanged at certain time periods, for instance, if broken or if the capacity has gone down due to filling of the one or more filters. Also, one or more UVC lamps may be broken and needs exchange, which means separating the air sterilizer unit. In view of this the present invention also concerns a filter housing having an interior space wherein at least one UVC light source adapted for radiating microorganisms, is located, and at least one filter with a capacity to reduce or remove air borne particles and microorganisms from ambient air, wherein the filter is UVC resistant and is located between the UVC light source and the at least one outlet. It is intended that all relevant embodiments described below in relation to the filter housing of the air sterilizer unit are also embodiments of the filter housing when not connected to the air transportation unit.

Following this the present invention relates to a kit of parts comprising an air sterilizer unit having at least one inlet and at least one outlet when assembled, comprising in two parts:

(a) a filter housing having an interior space wherein at least one UVC light source adapted for radiating microorganisms, is located, and at least one filter with a capacity to reduce or remove air borne particles and microorganisms from ambient air, wherein the filter is UVC resistant and is located between the UVC light source and the at least one outlet; and

(b) an air transportation unit, wherein the air transportation unit is adapted to move ambient air from the at least one inlet into the filter housing and being exposed to the UVC light and through the at least one filter, and through the at least one outlet.

In one embodiment of the present invention the air sterilizer unit wherein a pre-filter capable of removing air borne particles having a diameter less than 200 μm is located at the inlet. Preferably, the pre-filter is capable of removing air borne particles having a diameter less than 100 μm, such as between 10 and 100 μm. Typically, the pre-filter is a class G3 filter with Arrestance A (5) 80<A<90. Examples of particles to be removed are drops, dirt, visible dust.

In a further embodiment of the present invention the air transportation unit comprises a fan having sufficient capacity to move ambient air into the filter housing and through the at least one filter and the at least one outlet.

In a further embodiment of the present invention the UVC light source is adapted to provide UVC light at 100-280 nm, such as from 250-260 nm, such as 254 nm. The UVC light at 250-260 nm provides a much more effective killing of bacteria and virus while at the same time the UCV light does not destroy the material of the air sterilizer unit, in particular the various filters as defined.

In a further embodiment of the present invention the at least one filter is a HEPA filter capable of removing air borne particles having a diameter less than 0.5 μm. Preferably, the HEPA filter is a HEPA14 filter capable of removing 99,995% of air borne particles 0.3 micrometers (μm) in diameter. HEPA stands for high efficiency particulate air and is sometimes also referred to as high-efficiency particulate arresting or high-efficiency particulate arrestance.

In a further embodiment of the present invention a second filter which is UVC resistant is located between the UVC light source and the at least one filter and comprises an active carbon filter capable of removing air borne particles having a diameter less than 100 μm, such as between 10 and 100 μm. Typically, the carbon filter is a class G4 filter with Arrestance A (5) 90<A. Examples of particles are visible dust. Typically, the carbon filter has a surface area of between 0.5 to 2 m², such as from 0.2 to 1.5 m². When the second filter is present the ambient air to be cleaned first travels into the filter housing where it is radiated and the through the second filter and then through the at least first filter and out via the at least one outlet.

In a further embodiment of the present invention a third filter which is UVC resistant is located between the UVC light source the second filter and capable of removing air borne particles having a diameter less than 100 μm, such as between 10 and 100 μm, wherein the filter is coated with TiO2. Typically, the third filter is a glass filter which is coated with TiO2, such as a class MS filter with efficiency of E (%) 40<E<60. Examples of particles to be removed are visible dust and pollen. Typically, the TiO₂ coated filter has a surface area of between 0.1 to 1.5 m², such as from 0.2 to 0.5 m². When the third filter is present the ambient air to be cleaned first travels into the filter housing where it is radiated and then through the third filter, and hereafter travels through the second filter and then through the at least first filter and out via the at least one outlet.

The term “arrestance” as used herein is a measure for how efficient the filter removes larger particles.

In a further embodiment of the present invention the filter housing is a cylinder shaped housing. Typically, the air sterilizer unit is adapted to draw ambient air into the air transportation unit and into the filter housing to receive radiation by the UVC light and out through the at least one outlet located at the side of the cylinder housing. Other constructions of such a filter house is possible such as an ellipse shaped construction. In a typical embodiment the cylinder shaped housing is a cartridge filter housing.

In a further embodiment of the present invention the UVC light source comprises between 1 and 10 UVC lamps. In reality only, the size of the available space inside the filter housing sets the limits for UVC lamps, but for a stand-alone unit, it is preferable to have from 1-10 lamps. In one embodiment the UVC light source is 3 UVC lamps. In another embodiment the UVC light source is 4 UVC lamps. Preferably, the UVC lamps are located so that the distance between each light source is the same.

In a further embodiment of the present invention the HEPA filter is folded, such as pleated, to create a large surface area. Typically, the HEPA filter is folded to create a surface area of between 1 to 20 m², such as from 5 to 15 m².

In a further embodiment of the present invention the air sterilizer unit is a stand-alone device.

In a further embodiment of the present invention the air sterilizer unit as a stand-alone device is suitable for functioning inside a closed room.

In another aspect the present invention relates to a method of removing microorganisms from ambient air comprising supplying current to an air filter having at least one inlet and at least one outlet comprising

(a) a filter housing having an interior space wherein at least one UVC light source adapted for radiating microorganisms, is located, and at least one filter with a capacity to reduce or remove microorganisms from ambient air, wherein the filter is UVC resistant and is located between the UVC light source and the at least one outlet,

(b) an air transportation unit, wherein the air transportation unit is adapted to move ambient air from the at least one inlet into the filter housing, and being exposed to the UVC light and through the at least one filter, and through the at least one outlet; and moving ambient air from the at least one inlet into the filter housing, wherein the air is being exposed to the UVC light and further moving the air exposed to UVC light through the at least one filter, and through the at least one outlet, wherein microorganisms and other particulate material have been removed from the air leaving the at least one outlet.

In a further aspect the present invention relates to a method of removing microorganisms from ambient air comprising supplying current to the air sterilizer unit of the present invention and any one of the defined embodiments and moving ambient air from the at least one inlet into the filter housing, wherein the air is being exposed to the UVC light and further moving the air exposed to UVC light through the at least one filter, and through the at least one outlet, wherein microorganisms and other particulate material have been removed from the air leaving the at least one outlet.

In another aspect the present invention relates to a method of removing microorganisms and other particulate material from ambient air comprising supplying current to the air sterilizer unit of the present invention and any one of the defined embodiments and moving ambient air from the at least one inlet into the filter housing, wherein the air is being exposed to the UVC light and further moving the air exposed to UVC light through the at least one filter, and through the at least one outlet, wherein microorganisms and other particulate material have been removed from the air leaving the at least one outlet.

In a further aspect the present invention relates to use of an air filter having at least one inlet and at least one outlet comprising

(a) a filter housing having an interior space wherein at least one UVC light source adapted for radiating microorganisms, is located, and at least one filter with a capacity to reduce or remove microorganisms from ambient air, wherein the filter is UVC resistant and is located between the UVC light source and the at least one outlet,

(b) an air transportation unit, wherein the air transportation unit is adapted to move ambient air from the at least one inlet into the filter housing, and being exposed to the UVC light and through the at least one filter, and through the at least one outlet;

in a room, for removing microorganisms from the room.

In a further aspect the present invention relates to use of an air sterilizer unit of the present invention and any one of the defined embodiments in a room, for removing microorganisms from the room.

In a further aspect the present invention relates to use of an air sterilizer unit of the present invention and any one of the defined embodiments in a room, for removing microorganisms and other particulate material from ambient air from the room.

The term “stand-alone” as used herein means a device which can be transported by the user, such as people working at a hospital, and plugged-in (for current supply) at any desired place, such as in a hospital room for sick people, a room for surgery, an office in a building.

The invention will now be described more fully with reference to the appended drawings illustrating typical embodiments of the air sterilizer unit of the present invention.

These drawings are by no means limiting the scope of the present invention and are only intended to guide the skilled person for better understanding of the present invention.

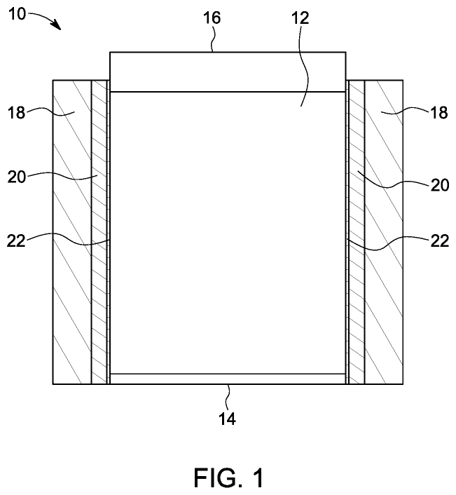

FIG. 1 illustrates a cross/side section side-view of a cartridge filter device (10) having an interior space or chamber (12) wherein at least one UVC is located (not shown). The filter device has a cylindric house with a top (14) closed and non-permeable to air, and a bottom flange (16) which is adapted to receive an air transportation unit in accordance with the present invention. The outer circumference of the filter device (10) is a HEPA filter (18) which can remove microorganisms. In close proximity and surrounded by the first filter (18) is a second carbon filter (20) which can remove odors and other particulate material, when the air is pushed through. The second filter (20) is in close proximity to a third filter (22) typically a glass filter coated with TiO₂, and surrounds such filter (22). The third filter (22) surrounds a space (12) and is the filter that is directly exposed to UVC light when the device (10) is in operation.

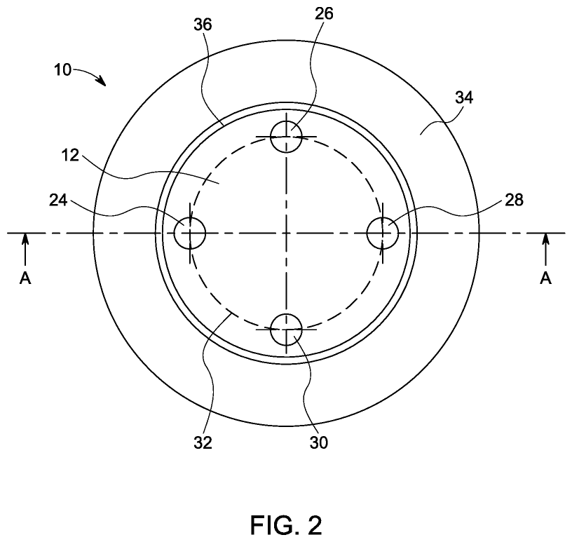

FIG. 2 illustrates a top view cross section of the cartridge filter device (10), and shows its cylindrical shape. Inside the cartridge filter device (10) is a space or chamber (12), wherein 4 UVC lamps (24, 26, 28, 30) are located with the same distance from each other and placed on a circumference (32). As explained in FIG. 1 the first filter, typically a HEPA14 filter, and optionally a second carbon filter (34) defines an outer circumference and a third filter (36) defines the inner circumference and surrounds the space (12).

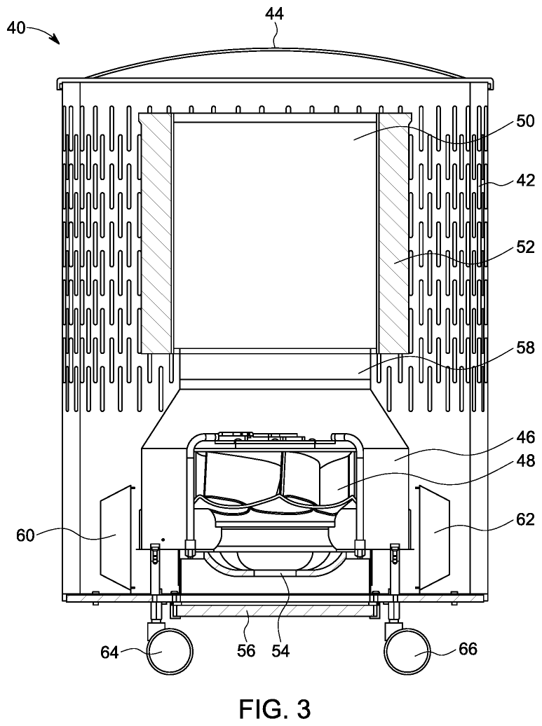

FIG. 3 illustrates an embodiment of the cartridge filter device of the present invention (40), protected by a cover panel (42) having a top cover (44) and wherein the cover panel (42) extends down to protect an air transportation unit (46) such as a fan house having a fan (48) adapted to draw air from the surroundings into the device (40). Inside the cover panel (42) is the cartridge filter device and the fan house (46) with the fan (48) wherein the fan when in operation draws ambient air into the chamber (50) and out through the one or more filters (52) and further out a metal mesh (not shown) at the sides of the cartridge filter (40). UVC lamps (not shown) are located inside the filter chamber (50). The bottom flange (58) of the cartridge filter house fits into a corresponding flange with the fan chamber (46) holding the two pieces together in air tight mode. Ballasts (60, 62) are placed on each side of the fan house (46) and may be facing the fan house and/or may be facing the cover panel (42). At the bottom of the fan house (46) is a flow grid (54) and ambient air is being drawn into the fan house (46) through a pre-filter (56). The cartridge filter device (40) is equipped with wheels (64, 66) to make it easy to move for a person working at for instance a hospital.

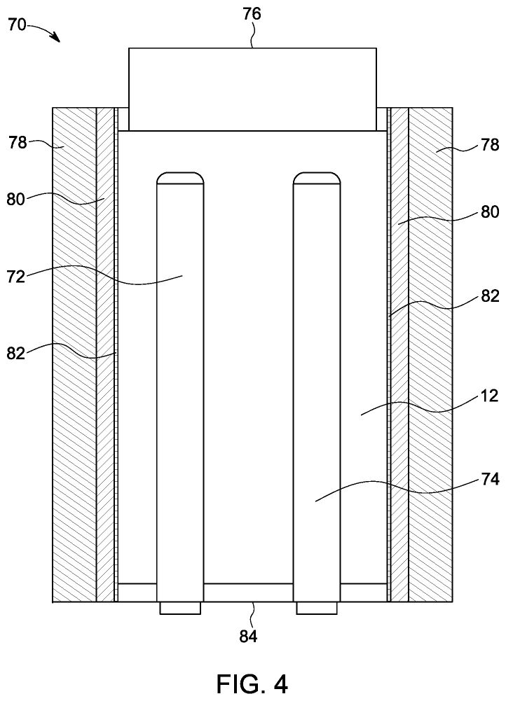

FIG. 4 illustrates a side-view cross section of the cartridge filter (70) of the present invention, wherein the UVC lamps (72, 74) are shown. The filter (70) has an interior chamber (12) wherein at least one UVC is located (72, 74). The filter device has a cylindric house with a top (84) closed and non-permeable to air, and a bottom flange (76) which is adapted to receive an air transportation unit in accordance with the present invention. The outer circumference of the filter device (70) is typically a HEPA filter (78) which can remove microorganisms. In close proximity and surrounded by the first filter (78) is a second carbon filter (80) which can remove odors and other particulate material, when the air is pushed through. The second filter (80) is in close proximity to a third filter (82) typically a glass filter coated with TiO₂ and surrounds such filter (82). The third filter (82) surrounds the space (12) and is the filter that is directly exposed to UVC light (72, 74) when the device (70) is in operation.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

Recitation of ranges of values herein are merely intended to serve as a short method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about”, where appropriate).

All methods described herein can be performed in any suitable order unless otherwise indicated herein or otherwise clearly contradicted by context.

The terms “a” and “an” and “the” and similar referents as used in the context of de-scribing the invention are to be construed to insert both the singular and the plural, unless otherwise indicated herein or clearly contradicted by context. Thus, “a” and “an” and “the” may mean at least one, or one or more.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

Throughout the description when “selected from” or “selected from the group consisting of” is used it also means all possible combinations of the stated terms, as well as each individual term.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The features disclosed in the foregoing description may, both separately and in any combination thereof, be material for realizing the invention in diverse forms thereof.

### EXPERIMENTALS

The air sterilizer unit of the present invention as a cartridge filter device (see FIGS. 1-3) have been made in two versions, a smaller version with an air intake of 500 m³/hour during the day and 250 m³/hour during the night, and a larger version with an air intake of 1000 m³/hour during the day and 500 m³/hour during the night.

The smaller version has a 6.7 m² HEPA 14 filter, a 1.0 m² carbon filter and a 0.28 m² TiO₂ coated filter. Also, there are three UVC lamps.

The larger version has a 14.2 m² HEPA 14 filter, a 1.554 m² carbon filter and a 0.42 m² TiO₂ coated filter. Also, there are four UVC lamps.

Both versions when current is applied operates by taking a large amount of ambient air (see details above) into the cartridge filter device, and by means of a rotating fan (backward curved centrifugal fan; B-wheel) the ambient air is further pushed through the different filters and out through the sides of the cartridge filter device.

The large cartridge filter device has a diameter of 470 mm and a height of 400 mm. In one version this filter is composed of three filter materials, wherein one is a HEPA14 filter with a surface area of 14.2 m², the second is a 1.554 m² carbon filter and the third is a glass based filter coated with 0.28 m² TiO₂. There are 4 UVC lamps each with a radiation wavelength of 254 nm.

It has been observed that the use of a glass based filter coated with TiO₂ leads to higher efficiency in killing microorganisms, and without being bound by theory it is contemplated that when titanium dioxide (TiO₂) is irradiated with UVC light a photocatalytic reaction takes place. Strong oxidizing hydroxyls radicals is formed. The radicals destroy the cell wall of the bacteria. Titanium dioxide (TiO₂) function as a catalyst in the process and is therefore not consumed over time.

In the present experiment such cartridge filter is placed in a hospital room wherein ambient air is drawn into the cartridge filter from the bottom through a pre-filter (class G3) comprising polyester fibers. Hereafter, the air is lead through the ventilator house with the centrifugal fan (air transportation unit) and further up and entering the space of the cartridge filter wherein the UVC lamps radiates the air and kills microorganisms. The air wherein microorganisms and other pollutant particles have been removed then returns to the room as clean and sterilized air.

In another experiment a cartridge filter having three filters is tested. As explained above the air is drawn into the space of the cartridge filter wherein the UVC lamps radiates the air and kills microorganisms. There are 4 UVC lamps producing a radiation wavelength of 254 nm, and each lamp has an intensity of 98 μW/cm² and the total intensity is 392 μW/cm². After being radiated the air is first lead through a glass based filter coated with TiO₂, and then the air is pushed through a second filter based on active carbon which absorbs odors and other particulate pollutants Finally, the air is pushed through the HEPA14 filter which is folded/pleated to remove microorganisms and has a filtration efficiency of 99.995% of airborne particles (bacteria and virus) of 0.3 μm in diameter. The cleaned air is then recirculated into to surrounding space.

Experiments with this cartridge filter device to test the active carbon filter gave the following results, wherein efficiency is indicated by numbers 1 to 4.

Table showing efficiency of activated carbon filter for various agents.

| | | | | | |
|---|---|---|---|---|---|
| acetone | 3 | Fatty acids | 4 | carbon dioxide CO2 | — |
| Acetaldehyde | 4 | chlorin | 1 | chemical solvent | 4 |
| acrolein | 1 | chloroform | 4 | menthol | 4 |
| alcohol | 4 | diesel oil mist | 3 | methane | 1 |
| anaesthetics | 3 | acetic acid | 4 | methanol | 3 |
| ether | 3 | disinfectant | 4 | merkaptane | 2 |
| essential oil | 4 | formaldehyd | 2 | phenol | 4 |
| ethane | 1 | Fruit odor | 4 | phosgene | 3 |
| ethylen | 1 | Household odor | 4 | propane | 2 |
| ethyl acetate | 4 | iodine | 4 | Candy smell | 4 |
| amines | 2 | cerosine | 4 | tetrachlorcarbons | 4 |
| ammonia | 1 | sweat | 4 | turpentine | 4 |
| gasoline | 4 | cosmetic | 4 | Tobacco odor | 4 |
| benzene | 4 | hospital odor | 4 | toluol | 4 |
| buthane | 2 | cresol | 4 | | |

4 = very good adsorption, 3 = good adsorption, 2 = acceptable adsorption, 1 = reduced adsorption

The ability to handle large amounts of air (1.000 m³/hour) demands a large filter area and by folding as a pleated filter a very large surface area of 14.2 m² have been created in this large cartridge filter device having a diameter of 450 mm and a height of 400 mm Experiments with an air sterilizer unit (cartridge filter) having a diameter of 560 mm and a height of 560 mm of the present invention with 4 UVC lambs producing a radiation wavelength of 254 nm and wherein the filter for reducing microorganisms had a surface area of 20.16 m2 and is a M6 with efficiency E (%) 60<E<80 (removing particles with diameter of 10-25 μm) showed 30-40% reduction in microbial counts compared to the situation without filter but regular cleaning.

1-15. (canceled)

16. An air sterilizer unit having at least one inlet and at least one outlet comprising:
(a) a filter housing having an interior space wherein at least one UVC light source adapted for radiating microorganisms, is located, and at least one filter with a capacity to reduce or remove microorganisms from ambient air, wherein the filter is UVC resistant and is located between the UVC light source and the at least one outlet, and
(b) an air transportation unit, wherein the air transportation unit is adapted to move ambient air from the at least one inlet into the filter housing and being exposed to the UVC light and through the at least one filter, and through the at least one outlet.

17. The air sterilizer unit of claim 16, wherein a pre-filter capable of removing air borne particles having a diameter less than 200 μm, is located at the inlet.

18. The air sterilizer unit of claim 16, wherein the air transportation unit comprises a fan having sufficient capacity to move ambient air into the filter housing and through the at least one filter and the at least one outlet.

19. The air sterilizer unit of claim 16, wherein the UVC light source is adapted to provide UVC light at 250-260 nm.

20. The air sterilizer unit of claim 16, wherein the at least one filter is a HEPA filter capable of removing air borne particles having a diameter less than 0.5 μm.

21. The air sterilizer unit of claim 16, wherein a second filter which is UVC resistant is located between the UVC light source and the at least one filter and comprises an active carbon filter capable of removing air borne particles having a diameter less than 100 μm.

22. The air sterilizer unit of claim 16, wherein a third filter which is UVC resistant is located between the UVC light source and the second filter and is capable of removing air borne particles having a diameter less than 100 μm, wherein the filter is coated with TiO₂.

23. The air sterilizer unit of claim 16, wherein the filter housing is a cylinder shaped housing.

24. The air sterilizer unit of claim 16, wherein the UVC light source is between 1 and 10 UVC lamps, such as 3-4 UVC lamps.

25. The air sterilizer unit of claim 23 adapted to draw ambient air into the air transportation unit and into the filter housing to receive UVC light and out through the at least one outlet located at the side of the cylinder housing.

26. The air sterilizer unit of claim 16, wherein the HEPA filter is folded, to create a large surface area.

27. The air sterilizer unit of claim 16 which is a stand-alone device.

28. The air sterilizer unit of claim 16 which is suitable for functioning inside a closed room.

29. A method of removing microorganisms from ambient air comprising supplying current to the air sterilizer unit of claim 16 and moving ambient air from the at least one inlet into the filter housing, wherein the air is being exposed to the UVC light and further moving the air exposed to UVC light through the at least one filter, and through the at least one outlet, wherein microorganisms have been removed from the air leaving the at least one outlet.

30. Use of an air sterilizer unit of claim 16 in a room, for removing microorganisms from the room.
